(19) United States
(12) Patent Application Publication
(10) Pub. No.: US 2021/0236519 A1
(43) Pub. Date: Aug. 5, 2021

(54) CONTROLLED RELEASE DOSAGE FORMS OF 5-AMINOSALICYLIC ACID AND PROCESS THEREOF

(71) Applicant: ATOZ PHARMACEUTICALS PVT LTD, Chennai (IN)

(72) Inventors: OLAGANATHAN ARUMUGAM, Chennai (IN); NATARAJAN VENKATACHALAM, Chennai (IN)

(21) Appl. No.: 17/163,358

(22) Filed: Jan. 30, 2021

(30) Foreign Application Priority Data
Jan. 30, 2020 (IN) 202041004130

(51) Int. Cl. A61K 31/606; A61K 9/28; A61K 9/20

(52) U.S. Cl. CPC A61K 31/606; A61K 9/2866; A61K 9/2893; A61K 9/284; A61K 9/2873; A61K 9/2813; A61K 9/282; A61K 9/2095

(57) ABSTRACT

Accordingly, the invention provides controlled release minitablets of 5-ASA with high drug load and less net weight for easier administration. In another aspect of the present invention, the process of preparation of 5-ASA or its prodrugs/derivatives as coated minitablets, wherein the minitablets are prepared by wet granulation of 5-Aminosalicylic acid or its prodrugs/derivatives and at least one pharmaceutical retarding agent either at intra granular or extragranular stage and at least one pharmaceutical excipient or carrier of other categories for tableting. Then, the granules are size controlled through milling with size in the range of 100 microns to 700 microns. Further the granules are compressed using single tip or multi-tip punch and die to get the required size and shape and used as minitablets in sachets for administering with water/soft foods or encapsulated in capsules for whole administration.

### FIELD OF THE INVENTION

The present invention relates to the formulation of controlled release 5-Aminosalicylic acid (5-ASA) or its prodrugs or derivatives thereof as minitablets with rate controlling film coating in less net weight than conventionally available controlled release dosage forms. The present disclosure also provides for a composition of minitablets, coating, and process for preparation of controlled release film coated minitablet. The present application is based on, and claims priority from an Indian Application Number 202041004130 filed on 30 Jan. 2020 the disclosure of which is hereby incorporated by reference herein

### BACKGROUND OF THE INVENTION

5-Aminosalicylic acid (5-ASA) or its prodrugs/derivatives thereof are the first line therapy for the treatment of ulcerative colitis, an inflammatory bowel disease—characterized by chronic relapsing inflammation in the colon and rectum. The mechanism of action of 5-ASA is unknown but appears to be topical rather than systemic. Mucosal production of arachidonic acid metabolites, both through the cyclooxygenase pathways, that is, prostanoids, and through the lipoxygenase pathways, that is, leukotrienes and hydroxy eicosatetraenoic acids, is increased in patients with chronic ulcerative colitis, and it is possible that mesalamine diminishes inflammation by blocking cyclooxygenase and inhibiting prostaglandin production in the colon. (https://www.accessdata.fda.gov/drugsatfda_docs/label/2014/204412s003lbl.pdf)

5-ASA dosage ranges from 1.5 g/day to the maximum of 4.8 g/day depend upon its usage in induction or remission. The other known prodrugs or derivatives of 5-ASA approved for the indications for the treatment of inflammatory bowel disease such as ulcerative colitis are balsalazide, olsalazine, and sulfasalazine. Dosage of balsalazide starts with minimum of 2.25 g/day for paedriatics to maximum of 6.75/day for adults. The usual dosage of olsalazine in adults is 1.0 g/day and the sulfasalazine for initial therapy is 3-4 g/day and for maintenance therapy is 2 g/day. The dosage of sulfasalazine is adjusted according to the individual response and tolerance.

As stated above, dosage levels, number of units, unit size to be administered for clinical efficacy in ulcerative colitis makes the dosage administration monotonous for paedriatics, adults and elderly patients. As per the size, shape, and other physical attributes of tablets and capsules guidance by FDA, over 16 million people in the United States have some difficulty swallowing, also known as dysphagia. A survey of adults on difficulties on swallowing of the tablets and capsules suggests that this problem goes well beyond the patient population with clinically recognized dysphagia and may affect as many as 40 percent of Americans. Of those who experience difficulty swallowing medications, less than a quarter discuss the problem with a health care professional, 8 percent admit to skipping a dose of prescribed medication, and 4 percent have discontinued therapy because the tablets and/or capsules were difficult to swallow. Individuals who find it difficult to swallow tablets and capsules frequently cite the size as the main reason for the difficulty in swallowing.

Other adverse events such as pain, gagging, choking, and aspiration are related to swallowing difficulties in the oropharyngeal phase of swallowing and increasingly occur at larger tablet and capsules. Children and adolescents, as well as the elderly, are more likely to have difficulty swallowing tablets or capsules.

From the approved products (List below) for oral administration, Pentasa™ 250 and 500 mg and Colazal™ 750 mg is available as beads (pellets) in capsules with the advantage of sprinkling in apple sauce for peoples who are difficult to swallow or either for paedriatic or geriatric treatments. The other high strength products (dosage forms) are not recommended to be sprinkled. A comprehensive list of approved drugs for treatment of ulcerative colitis/inflammatory bowel disease, their dosage forms, and their dosage strengths are herewith listed below in Table 1.

TABLE 1

| | | | | Approx. | |
|---|---|---|---|---|---|
| | Dosage | | | Size of | |
| Product | Form | | | Dosage | Pediatric |
| (Drug) | Description | Strength | | form | Indication |
| Pentasa | Pellets in | 250 & 500 | | 22 mm | No |
| (Mesalamine) | Capsule | mg | | 25 mm | 18 years or |
| | | | | | older |
| Apriso | Pellets in | 375 | mg | 23 mm | No |
| (Mesalamine) | Capsule | | | | 18 years or |
| | | | | | older |
| Lialda | Tablet DR | 1.2 | gm | 20 mm | No |
| (Mesalamine) | | | | | |
| *Asacol | Tablet DR | 400 | mg | 14 mm | No |
| (Mesalamine) | | | | | |
| Asacol HD | Tablet DR | 800 | mg | 19 mm | No |
| (Mesalamine) | | | | | 18 years or |
| | | | | | older |
| Delzicol | 4 DR tablets | 400 | mg | 22 mm | Yes |
| (Mesalamine) | in a capsule | | | | 5 Years and |
| | | | | | older |
| Dipentum | Capsule | 250 | mg | 19 mm | No |
| (Olsalazine) | | | | | |
| *Giazo | Tablet | 1.1 | g | 19 mm | No |
| (Balsalazide) | | | | | 18 years or |
| | | | | | older (M) |
| Colazal | Capsule | 750 | mg | 23 mm | Yes |
| (Balsalazide) | | | | | 5 years and |
| | | | | | older |
| Azulfidine | Tablet | 500 | mg | 18 mm | Yes |
| (Sulfasalazine) | | | | | 6 years and |
| | | | | | older |
| *Azulfidine | Suspension | 250 mg/5 ml | | Not | Yes |
| (Sulfasalazine) | | | | Applicable | 6 years and |
| | | | | | older |
| Azulfidine EN | Tablet, | 500 | mg | 18 mm | Yes |
| (Sulfasalazine) | Delayed | | | | 6 Years and |
| | release | | | | older |

*Discontinued from the market.

Marketed 5-ASA product, Pentasa is prepared by drug layering on sugar-based core and then a functional coating. This process involves combination of many cost factors like more inactive ingredients, multistep and multiple process parameter control. The marketed Pentasa product containing 500 mg of 5-ASA is formulated with 300 mg excipient, accounting to the total weight of 800 mg and encapsulated in “00EL” Size capsule.

Although not all patient factors can be addressed through pharmaceutical design and manufacture, the physical characteristics of a product can be addressed to improve the patient compliance. These physical characteristics influence not only vulnerable populations like pediatrics, elders and others who have dysphagia but also the ability of certain adults to swallow the product when its administered whole. The product that can be effectively developed and manufactured to minimize swallowing difficulties, will encourage and improve patient compliance with medication regimens of larger dose and dosage forms. Improving drug load with less amount of excipients helps in reducing the quantum or size of final dosage form.

The development of 5-ASA or its prodrugs/derivatives as controlled release minitablets in net less weight with its required pharmacokinetic functionality, sprinkling option is one of the challenging physical characteristics requirement for every mesalamine administrating population including pediatric, geriatric and dysphagia. In addition, formulation of 500 mg 5-ASA as Controlled release minitablets with less excipient load to accommodate in capsules with size not more than “00-Size” is a challenging task.

The present invention is to prepare 5-ASA or its prodrugs/derivatives as minitablets through the composition devoid of sucrose, non-pareil seeds and the process free of extrusion and spheronization technique. The invention also provides core minitablets of high drug load for controlled (sustained/prolonged/timed) release (CR) with controlled release coating.

The above information is presented as background information only to help the reader to understand the present invention. Applicants have made no determination and make no assertion as to whether any of the above might be applicable as prior art with regard to the present application.

### OBJECT OF THE INVENTION

The principal object of the present invention is to get more convenient, high patient compliance by providing the 5-ASA or its prodrugs/derivatives as minitablets in less net weight than conventionally available controlled release dosage forms of 5-ASA or its prodrugs/derivatives thereof with minimal excipients.

Another object of the invention is to provide a solid minitablet with largest dimension is lesser or equal to 3.0 mm of 5-ASA or its prodrugs/derivatives as minitablets.

Another object of this invention is to provide for a simple composition and process of preparation for 5-ASA or its prodrugs/derivatives as minitablets.

Yet another object of present invention is to provide composition of functional coating of minitablets of 5-ASA or its prodrugs/derivatives for controlled release (CR).

### SUMMARY OF THE INVENTION

Accordingly, the present invention provides for a controlled release composition having an in vitro release profile where the drug release in pH 4.5 is between 50-60% of the active ingredient (5-ASA or a pharmaceutically acceptable salt, solvate, and/or ester) at 12^{th} hour and in pH 7.5 is between 90-100% at 6^{th} hour. The controlled released composition comprising: a) a core minitablet comprising: i) 5-ASA or a pharmaceutically acceptable salt, solvate, and/or ester thereof having a weight percentage in a range of 80%-90% w/w with respect to the core weight of the core mini tablet; and b) a coating layer comprising: i) hydrophobic cellulose to the weight gain of 0.5% to 1%; or ii) hydrophobic cellulose and hydrophilic polymer mixture, coated to weight gain of 2%-10% to the total weight of the core minitablet, such that the total weight of the controlled release composition is in the range of 595-650 mg. The net weight of the controlled release composition of the present invention is much lower compared to the conventionally available drug formulations of mesalamine available in the market, thereby minimizing swallowing difficulties, and improve patient compliance and overcome the challenges associated with medication regimens of larger dose and dosage forms.

In another aspect of the present invention, the controlled release minitablets dissolution profile in pH 0.1N HCl, pH 4.5, and pH 7.5 is as follows in Table 1.

TABLE 1
Amount of drug dissolved (%) Limits as per Reference Vs Time

| Hours | 0.1N HCL | pH 4.5 | pH 7.5 |
|---|---|---|---|
| 1 | 30-50 | 5-15 | 5-30 |
| 2 | 50-75 | 8-18 | 30-60 |
| 4 | 75-95 | 18-28 | 60-80 |
| 6 | Not Less than 90% | 28-38 | 80-100 |
| 8 | | 38-48 | |
| 12 | | 50-60 | |

In another aspect of the present invention, the process of preparation of 5-ASA or its prodrugs/derivatives as minitablets, wherein the minitablets is prepared by at least of any of the granulations techniques like wet granulation, dry granulation and or hot melt granulation of 5-Aminosalicylic acid or its prodrugs/derivatives with any pharmaceutical acceptable binder or with excipient of low melting point or both. Then, the granules are size controlled through mill and/or sifter of any mesh range between mesh size #16 and mesh #60. Further the granules are compressed using single tip or multi-tip (of about maximum 16 tips and die to get the required size and shape) punches.

These and other aspects of the embodiments herein will be better appreciated and understood when considered in conjunction with the following description. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### DETAILED DESCRIPTION OF INVENTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. Also, the various embodiments described herein are not necessarily mutually exclusive, as some embodiments can be combined with one or more other embodiments to form new embodiments. The term “or” as used herein, refers to a non-exclusive or, unless otherwise indicated. The examples used herein are intended merely to facilitate an understanding of ways in which the embodiments herein can be practiced and to further enable those skilled in the art to practice the embodiments herein. Accordingly, the examples should not be construed as limiting the scope of the embodiments herein.

Conventional dosage forms of 5-ASA or its or its pharmaceutically acceptable salt, solvate, and/or ester thereof are larger in size due to its dosage levels. Till today, the market available dosage form of 5-ASA in tablet forms are more than 18 mm in its largest dimension, and have a net weight greater than 800 mg. 5-ASA or its prodrug/derivatives in capsule forms are in the size range of 19 mm to 25 mm in its largest dimension. Also, these drugs are administered as multiple units at a time according to the clinical requirement or severity in fewer available capsules (treatment or maintenance for ulcerative colitis) due to its high dose or dosage level. Moreover, marketed products have excess excipients added either as inert diluent or startup sugar or other pharmaceutical acceptable startup beads (or spheres) or more percentage of coating layering affects the final dosage size of the product i.e. the encapsulating gelatin shells or capsule size. Moreover, this in turn generates a fear for administering the bigger size dosage forms. In order to circumvent the bigger size, the object of the present invention is to provide unique combination of composition and the process for the preparation of controlled release minitablets to reduce the net weight thereby the size of the final dosage form its encapsulating, e.g. capsules.

The controlled released composition comprising: a) a core minitablet comprising: i) 5-ASA or a pharmaceutically acceptable salt, solvate, and/or ester thereof having a weight percentage in a range of 80%-90% w/w with respect to the core weight of the core mini tablet; and b) a coating layer comprising: i) hydrophobic cellulose to the weight gain of 0.5% to 1%; or ii) hydrophobic cellulose and hydrophilic polymer mixture, coated to weight gain of 2%-10% to the total weight of the core minitablet, such that the total weight of the controlled release composition is in the range of 595-650 mg. In another embodiment, the hydrophobic cellulose is in the range of 0.5-1%. The net weight of the controlled release composition of the present invention is much lower compared to the conventionally available drug formulations of mesalamine available in the market, thereby minimizing swallowing difficulties, and improve patient compliance and overcome the challenges associated with medication regimens of larger dose and dosage forms.

In another embodiment of the present invention, the controlled released composition comprising: a) a core minitablet comprising: i) 5-ASA or a pharmaceutically acceptable salt, solvate, and/or ester thereof having a weight percentage in a range of 80%-90% w/w with respect to the core weight of the core mini tablet; and b) a coating layer comprising: i) hydrophobic cellulose to the weight gain of 0.5% to 1%; or ii) hydrophobic cellulose and hydrophilic polymer mixture, coated to weight gain of 2%-10% to the total weight of the core minitablet, and wherein ratio of the hydrophobic cellulose to the hydrophilic polymer mixture is in the range of 1:9 to 4:6, such that the total weight of the controlled release composition is in the range of 595-650 mg. In another embodiment, the ratio of the hydrophobic cellulose to the hydrophilic polymer mixture is 1:3. In another embodiment, the ratio of the hydrophobic cellulose to the hydrophilic polymer mixture is 4:6. In another embodiment, the ratio of the hydrophobic cellulose to the hydrophilic polymer mixture is 1:4.

In another embodiment of the present invention, the core minitablet further comprises binder(s) having a weight percentage in a range of 1%-10% with respect to the core minitablet; diluent(s) having a weight percentage in the range of 0.5%-20% with respect to the core minitablet, optionally disintegrant(s) having a weight percentage in a range of 1%-10% with respect to the core minitablet, lubricant(s) having a weight percentage in a range of 1%-5% with respect to the core minitablet, glidant(s) having a weight percentage in a range of 1%-5% with respect to the core minitablet, and retarding agent(s) having a weight percentage in the range of 4-18% with respect to the core minitablet.

In another embodiment of the present invention, the core minitablet comprising: 5-ASA or its pharmaceutically acceptable salt, solvate, and/or ester thereof having a weight percentage in a range of 80%-90% w/w with respect to the core weight of the core mini tablet; binder(s) having a weight percentage in a range of 1%-10% with respect to the core minitablet, wherein the binders are selected from a group consisting of polyvinyl pyrrolidine, hydroxypropyl cellulose, hypromellose, acacia, starch, starch derivatives and gelatin; the diluent(s) having a weight percentage in the range of 0.5%-20% with respect to the core minitablet, wherein the diluents are selected from a group consisting of lactose, lactose monohydrate, starch, hydrolysed starch, partially hydrolysed starch, stearic acid, microcrystalline cellulose, calcium phosphates, mannitol and/or any co-processed excipients of above; the disintegrant(s) having a weight percentage in a range of 1%-10% with respect to the core minitablet selected from a group consisting of crospovidone, sodium starch glycolate, starch, hydrolysed starch, partially hydrolysed starch, croscarmellose sodium; lubricant(s) having a weight percentage in a range of 1%-5% with respect to the core minitablet selected from a group consisting of magnesium stearate, calcium stearate, stearic acid, glyceryl dibehenate, glyceryl stearate, other glyceride derivatives, low MW polyethylene glycols; the glidant(s) having a weight percentage in a range of 1%-5% with respect to the core minitablet selected from the group consisting of colloidal silicon dioxide, silicon dioxide and talc; and retarding agent(s) having a weight percentage in the range of 4-18% with respect to the core minitablet selected from a group consisting of ethyl cellulose, magnesium stearate, calcium stearate, glyceryl monostearate, glyceryl dibehenate, glyceryl distearate, stearic acid and any stearic acid or fatty acid derivatives thereof.

In another embodiment of the present invention, the controlled release composition is optionally coated with a coating solution comprising seal coating polymers to bring about 0.3%-5% weight gain to the core minitablet. In another embodiment, the controlled release layer is coated on the core minitablet or a seal coated surface.

In another embodiment of the present invention, a method of preparing the pharmaceutical composition of claim 1, comprising: (a) granulating 5-ASA or a pharmaceutically acceptable salt, ester, and/or solvate thereof along with binders, retarding agents, and lubricants in a sequential manner to obtain the core minitablet; and (b) coating the core mini tablet with a coating layer comprising hydrophobic cellulose or mixture of hydrophobic cellulose and hydrophilic polymers.

In another embodiment of the present invention, the process to prepare the controlled release composition comprising: i) 5-ASA or a pharmaceutically acceptable salt, solvate, and/or ester thereof having a weight percentage in a range of 80%-90% w/w with respect to the core weight of the core mini tablet; and b) a coating layer comprising: i) hydrophobic cellulose to the weight gain of 0.5% to 1%; or ii) hydrophobic cellulose and hydrophilic polymer mixture, coated to weight gain of 2%-10% to the total weight of the core minitablet, such that the total weight of the controlled release composition is in the range of 595-650 mg, the method comprising: (a) mixing/granulating 5-ASA or premixed 5-ASA and retarding agent along with a pharmaceutically acceptable binder solution prepared in suitable solvents (e.g. water or ethanol or Isopropyl alcohol or mixture of two or more Solvent) to obtain a first mixture; b) drying the first mixture to a temperature range of 40-70° C. to obtain a dried mixture; c) passing the granules through mill and/or sifter of mesh range between mesh size #16 and mesh #60, wherein the granules have a size below 700 microns; d) mixing the granules obtained in step c) with other diluents, retarding agents, glidants and lubricants to obtain free flowing granule of 5-ASA; e) compressing the granules obtained in step d) using single tip or multi-tip (of about 16 tips) and die to obtain the core minitablet of claim 1; and f) coating the core minitablet with a coating solution comprising: i) hydrophobic cellulose to the weight gain of 0.5% to 1%; or ii) hydrophobic cellulose and hydrophilic polymer mixture, coated to weight gain of 2%-10% to the total weight of the core minitablet, such that the total weight of the controlled release composition is in the range of 595-650 mg.

A method to improve patient compliance comprising administering to a patient in need thereof the composition comprising: i) 5-ASA or a pharmaceutically acceptable salt, solvate, and/or ester thereof having a weight percentage in a range of 80%-90% w/w with respect to the core weight of the core mini tablet; and b) a coating layer comprising: i) hydrophobic cellulose to the weight gain of 0.5% to 1%; or ii) hydrophobic cellulose and hydrophilic polymer mixture, coated to weight gain of 2%-10% to the total weight of the core minitablet, such that the total weight of the controlled release composition is in the range of 595-650 mg.

In another aspect of the present invention, the composition to controlled release minitablets of 5-ASA or its prodrugs/derivatives comprising: a core minitablet and coating, wherein the core further comprises: i) any one 5-ASA drug or its pharmaceutically acceptable salt, solvate, and/or ester thereof, having a weight percentage in the range of 80%-90% w/w with respect to the core minitablet, ii) diluent(s) having a weight percentage of 0.5%-20% w/w with respect to the weight of the core minitablet, iii) binder(s) having a percentage of 1%-10% w/w with respect to the core minitablet, iv) release retarding agent(s) having a weight percentage of 4%-18% w/w with respect to the total weight of the core minitablet, wherein the retarding agent may be used in intragranular or extragranular or in both stages (either similar agents or mix match), v) lubricant(s) or glidant(s) or both having a weight percentage of 0.5%-5% w/w with respect to the weight of core minitablet. The coating layer further comprises i) functional coating of ethyl cellulose coating mixture or admixture of ethyl cellulose and immediate release coating agents to the weight gain of 0.3% to 10% to the weight of core minitablet ii) optionally one seal coating to the weight gain of 0.3%-5% w/w to the weight of core minitablet, wherein the coating solution is prepared from any pharmaceutically known conventional seal coat polymers.

In another aspect of present invention, the composition of coating or coating solution is mixture of ethyl cellulose and pharmaceutically known film coating material or water-soluble excipients dispersed or dissolved in solid ratio between 10:90 and 40:60 on total solid weight of coating material used.

In another aspect of present invention, the dissolution profile can be achieved using minitablets of either single mixture coating ratio or by combination of two or more different ratio.

In another aspect of present invention, the dissolution profile can be achieved using minitablets of similar size/shape or mixture of different sizes/shapes.

In another aspect of present invention, the dissolution profile can be achieved using similar or different size controlled release minitablets, wherein the minitablets are of similar release mechanism of same composition or different composition.

In another aspect of present invention, the minitablets can be encapsulated in capsules for whole administration or by opening and dispersing in water or by opening and spreading the minitablets in apple sauce or any soft foods.

In another aspect of present invention, the minitablets can be packed in sachets, wherein minitablets are to be administered by dispersing in water or by opening and spreading the minitablets in apple sauce or any soft foods.

In another aspect of present invention, the minitablets can be packed in multiple unit dose containers, wherein minitablets of respective unit dose prescribed are to be dispensed and taken either qualitatively (in a tablespoon or teaspoon) or quantitatively (in a volumetric device). The unit dose minitablets can be administered by dispersing in water or by opening and spreading the minitablets in apple sauce or any soft foods.

In another aspect of present invention, the minitablets can be suspended in syrup base, wherein suspension is to be administered by volume after shaking either qualitatively (in a tablespoon or teaspoon) or quantitatively (in a volumetric device).

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

### EXAMPLES

The disclosure will now be illustrated with working examples, which is intended to illustrate the working of disclosure not intended to take restrictively to imply any limitations on the scope of the present disclosure. Unless defined otherwise, all technical and scientific terms used herein have the same meaning as commonly understood to any one of the ordinary skilled in the art to which this disclosure belongs. Although methods and materials similar or equivalent to those described herein can be used in the practice of disclosed methods and compositions, the exemplary methods, devices and materials are described herein. It is to be understood that this disclosure is not limited to particular methods, and experimental conditions described, as such methods and conditions may apply.

### Example 1

Controlled release compositions: Various controlled release compositions, herewith referred to as compositions, from A1-A13 were prepared by mixing 5-ASA with other excipients like binders, diluents, retarding agents, glidants, lubricants in different w/w ratios. Further each of these compositions (A1-A11) were coated with various coating solutions and the effect of coating solutions on the dissolution profiles of these controlled release compositions are studied, and the results are provided under from Table 2 to 4. Compositions A12 and A13 are used as reference (devoid of any coating solution).

TABLE 2

| | A1 | A2 | A3 | A4 |
|---|---|---|---|---|
| Ingredients | (w/w %) | (w/w %) | (w/w %) | (w/w %) |
| 5-ASA (active | 81.3 | 84 | 87 | 86.8 |
| ingredient) | | | | |
| Glyceryl dibehenate | 9.8 | 6.7 | 3.5 | 8.7 |
| (retarding agent)-Intra | | | | |
| Granular | | | | |
| Polyvinylpyrrolidone | 0 | 6.7 | 7 | 0 |
| (binder) | | | | |
| Gelatin (binder) | 6.5 | 0 | 0 | 2.6 |
| Glyceryl dibehenate | 1.6 | 1.7 | 1.7 | 0 |
| (retarding agent and | | | | |
| lubricant) | | | | |
| Stearic acid | 0 | 0 | 0 | 1 |
| (retarding agent | | | | |
| and lubricant) | | | | |
| Colloidal | 0.8 | 0.9 | 0.8 | 0.9 |
| silicon dioxide | | | | |
| (glidant) | | | | |
| Water (Solvent) | q.s | q.s | q.s | q.s |
| Ethyl alcohol | 0 | 0 | 0 | 0 |
| (Solvent) | | | | |
| Total weight of | 615 | 595 | 575 | 576 |
| minitablets (mg) | | | | |
| Coating | 4.8% | 3.0% | 4.2% | 0.75% |
| | weight | weight | weight | weight |
| | gain of | gain of | gain of | gain |
| | surelease: | surelease: | surelease: | of diluted |
| | opadry II | opadry II | opadry II | surelease |
| | (40:60) to | (25:75) to | (25:75) to | (2%) |
| | the core | the core | the core | solution |
| | weight | weight | weight | to |
| | | | and | the core |
| | | | cured | weight |

TABLE 3

| | A5(w/ | A6(w/ | A7 (w/ | A8 (w/ |
|---|---|---|---|---|
| Ingredients | w %) | w %) | w %) | w %) |
| 5-ASA (active | 84 | 80.1 | 87 | 89.3 |
| ingredient) | | | | |
| Glyceryl dibehenate | 6.7 | 14.5 | — | — |
| (retarding agent)-Intra | | | | |
| Granular | | | | |
| Stearic acid (retarding | | | 3.7 | |
| agent)-Intra Granular | | | | |
| Polyvinylpyrrolidone | 6.7 | 2.4 | 7 | 0 |
| (binder) | | | | |
| Gelatin (binder) | 0 | 0 | 0 | 2.7 |
| Glyceryl dibehenate | 1.7 | 0 | 0 | 5.4 |
| (retarding agent and | | | | |
| lubricant) | | | | |
| Stearic acid (retarding | 0 | 2 | 1.7 | |
| agent and lubricant) | | | | |
| Magnesium Stearate | | | | 1.8 |
| Colloidal | 0.9 | 1 | 0.8 | 0.8 |
| silicon dioxide | | | | |
| (glidant) | | | | |
| Water (solvent) | q.s | q.s | — | q.s |
| Ethyl alcohol | 0 | 0 | q.s | 0 |
| (solvent) | | | | |
| Total weight of | 595 | 624 | 575 | 560 |
| minitablets (mg) | | | | |
| Coating | 5% weight | 1% weight | 3.25% | 1.5% |
| | gain of | gain of | weight | weight gain |
| | surelease: | aquarius | gain of | of diluted |
| | opadry II | to the | surelease: | surelease |
| | (50:50) | core | opadry II | (1%) |
| | to the | weight | (25:75) to | solution to |
| | core | | the core | the core |
| | weight | | weight | weight |

TABLE 4

| | | A10 | A11 | A12 | A13 |
|---|---|---|---|---|---|
| | A9 (w/w | (w/ | (w/ | (w/ | (w/ |
| Ingredients | %) | w %) | w %) | w %) | w %) |
| 5-ASA (active | 84 | 84 | 80 | 94.34 | 84 |
| ingredient) | | | | | |
| Glyceryl dibehenate | 6.7 | 6.7 | 9.6 | 0 | 6.7 |
| (retarding agent) | | | | | |
| Polyvinylpyrrolidone | 6.7 | 6.7 | 6.4 | 0 | 6.7 |
| (binder) | | | | | |
| Gelatin (binder) | 0 | 0 | 0 | 2.83 | 0 |
| Glyceryl dibehenate | 1.7 | 1.7 | 3.2 | 0 | 1.7 |
| (retarding agent) | | | | | |
| Stearic acid | 0 | 0 | 0 | 1.89 | 0 |
| (retarding agent and | | | | | |
| lubricant) | | | | | |
| Colloidal silicon | 0.9 | 0.9 | 0.8 | 0.94 | 0.9 |
| dioxide (glidant) | | | | | |
| Water (solvent) | q.s | q.s | q.s | q.s | q.s |
| Ethyl alcohol | 0 | 0 | 0 | 0 | 0 |
| (solvent) | | | | | |
| Total weight of | 595 | 595 | 625 | 530 | 595 |
| minitablets (mg) | | | | | |
| Coating | 8% | 12% | 2% | No | No |
| | weight | weight | weight | coating | coating |
| | gain of | gain of | gain of | (imme- | |
| | surelease: | surelease: | surelease: | diate | |
| | opadry II | opadry II | HPMC | release) | |
| | (20:80) to | (20:80) to | (40:60) to | | |
| | the core | the core | the core | | |
| | weight | weight | weight | | |

### Example 2

General process of preparing the controlled release compositions. The process steps include firstly mixing/granulating 5-ASA or 5-ASA and retarding agents by any of the granulation techniques known in the art (wet, dry or hot melt granulation) using a pharmaceutically acceptable binder to obtain a first mixture granule. The first mixture thus obtained was dried to a temperature range of 40-70° C. to obtain a dried mixture. The dried mixture were further passed through mill and/or sifter of mesh range between mesh size #16 and mesh #60 to obtain the granules of a size below 700 microns. Further, the milled granules was mixed with other excipients like diluents, retarding agents, glidants and lubricants to obtain final blend of 5-ASA. The final blend were compressed using single tip or multi-tip (of about 16) punch(s) and die to obtain the core minitablet of claim 1; and f) coating the core minitablet with a coating solution comprising: i) hydrophobic cellulose to the weight gain of 0.5% to 1%; or ii) hydrophobic cellulose and hydrophilic polymer mixture, coated to weight gain of 2%-10% to the total weight of the core minitablet, such that the total weight of the controlled release composition is in the range of 595-650 mg. The controlled release dosage forms are further cured (if required) and were analyzed for their drug release kinetics.

Process of preparation of A1: Minitablets of 2.5 mm was compressed and coated with surerelease:opadry II (40:60) as per the formula provided in Table 2. For this purpose, dry mix of mesalamine (also referred to as 5-ASA) with glyceryl dibehenate was carried out, followed by wet granulation in granulator using gelatin dissolved in water as binder, dried milled and then extra granulated with other excipients and lubricated. The coating is performed to achieve the target weight gain.

Process of preparation of A2: Minitablets of 2.5 mm was compressed and coated with surerelease:opadry II (25:75) as per the formula provided in Table 2. For this purpose, dry mix of mesalamine with glyceryl Dibehenate was carried out, followed by wet granulation in granulator using Polyvinylpyrrolidone dissolved in water as binder, dried, milled and then lubricated with respective lubricants. The coating is performed to achieve the target weight gain.

Process of preparation of A3: Minitablets of 2.0 mm was compressed and coated with surelease: opadry II as per the formula provided in Table 2. For this purpose, dry mix of mesalamine and glyceryl dibehenate was carried out, followed by wet granulation in granulator using polyvinylpyrrolidone as binder by dissolving in water, dried, milled and then mixed with glyceryl dibehenate and then with respective lubricants. The coating is performed to achieve the target weight gain and then cured.

Process of preparation of A4: Minitablets of 2.0 mm was compressed and coated with diluted surelease solution (2% w/w) as per the formula provided in Table 2. For this purpose, mesalamine granulation was carried out, by wet granulation in granulator using gelatin as binder by dissolving in water, dried, milled and then mixed with glyceryl dibehenate and then with respective lubricants. The coating is performed to achieve the target weight gain.

Process of preparation of A5: Minitablets of 2.0 mm was compressed and coated with Surelease: Opadry II (50:50) as per the formula provided in Table 3. For this purpose, dry mix of glyceryl dibehenate and mesalamine granulation was carried out, followed by wet granulation in granulator using polyvinylpyrrolidone dissolved in water as binder, dried, milled and then mixed with glyceryl dibehenate and then with respective lubricants. The coating is performed to achieve the target weight gain.

Process of preparation of A6: Minitablets of 2.0 mm was compressed and coated with Aquarius control ECD film coating solution as per the formula provided in Table 3. For this purpose, mesalamine along with glyceryl dibehenate was granulated by wet granulation in granulator using polyvinylpyrrolidone dissolved in water as binder, dried, milled and then mixed with stearic acid and then colloidal silicon dioxide. The coating is performed to achieve the target weight gain.

Process of preparation of A7: Minitablets of 2.5 mm was compressed and coated with sure release: Opadry II (25:75) as per the formula provided in Table 3. For this purpose, dry mix of mesalamine with stearic acid, followed by wet granulation in granulator using Polyvinylpyrrolidone as binder by dissolving in suitable organic solvent, dried and then lubricated with respective extragranular excipients and lubricants. The coating is performed to achieve the target weight gain.

Process of preparation of A8: Minitablets of 2.0 mm was compressed and coated with diluted surelease solution (1% w/w) as per the formula provided in Table 3. For this purpose, mesalamine granulation was carried out, by wet granulation in granulator using gelatin as binder by dissolving in water, dried, milled and then mixed with glyceryl dibehenate and then with respective lubricants. The coating is performed to achieve the target weight gain.

Process of preparation of A9: Minitablets of 2.0 mm was compressed and coated with Surelease: Opadry II (20:80) as per the formula provided in Table 4. For this purpose, mesalamine and glyceryl dibehenate premixed and granulation was carried out, by wet granulation in granulator using Polyvinylpyrrolidone as binder by dissolving in water, dried and then mixed with glyceryl dibehenate and then with respective lubricants. The coating is performed to achieve the target weight gain.

Process of preparation of A10: Minitablets of 2.0 mm was compressed and coated with Surelease: Opadry II (20:80) as per the formula provided in Table 4. For this purpose, mesalamine and glyceryl dibehenate premixed and granulation was carried out, by wet granulation in granulator using polyvinylpyrrolidone as binder by dissolving in water, dried and then mixed with glyceryl dibehenate and then with respective lubricants. The coating is performed to achieve the target weight gain.

Process of preparation of A11: Dry mix of Mesalamine with Glyceryl dibehenate was carried out, followed by wet granulation in granulator using polyvinylpyrrolidone as binder by dissolving in water, dried and then lubricated with extragranular excipients and respective lubricants. The coating is performed to achieve the target weight gain.

Process of preparation of A12—Core without retarding agents (immediate release): Mesalamine was granulated by wet granulation in granulator using gelatin as binder by dissolving in water, dried and then lubricated.

Process of preparation of A13—Core with retarding agent: Minitablets of 2.0 mm was compressed as per the formula below. For this purpose, mesalamine and glyceryl dibehenate premixed and granulation was carried out, by wet granulation in granulator using polyvinylpyrrolidone as binder by dissolving in water, dried and then mixed with glyceryl dibehenate and then with respective lubricants.

### Example 3—Dissolution Testing of Tablets

In vitro dissolution study conditions

| Strength of Dosage form | 500 mg |
|---|---|
| Apparatus | USP 2 (Paddles) |
| Speed | 100 rpm |
| Media | 0.1N HCL and Buffers at pH |
| | values 4.5 and 7.5 |
| Volume | 900 mL |
| Temperature | 37° C. |
| Sampling Time | 1, 2, 4, 6, 8 and 12 hours |

### Results and Discussion

The percentage release of mesalamine at 0.1 pH at various time intervals is herewith provided in Table 5.

TABLE 5

| Time | | | | | | | | | | | | | |
|---|---|---|---|---|---|---|---|---|---|---|---|---|---|
| (h) | A1 | A2 | A3 | A4 | A5 | A6 | A7 | A8 | A9 | A10 | A11 | A12 | A13 |
| 1 | 32 | 35 | 35 | 30 | 4 | 35 | 41 | 20 | 39 | 33 | 35 | 99 | 50 |
| 2 | 51 | 55 | 54 | 52 | 8 | 62 | 69 | 39 | 61 | 54 | 53 | 100 | 74 |
| 4 | 75 | 76 | 80 | 85 | 12 | 85 | 90 | | 81 | 77 | 80 | | 98 |
| 6 | 100 | 90 | 91 | 102 | | 103 | 102 | | 96 | 89 | 93 | | |
| 8 | | | 99 | | | 99 | | | 99 | 99 | 101 | | |
| 12 | | | | | | | | | | | | | |

From table 5 (A1-A11) it can be inferred that A1 to A11 met the dissolution criteria in 0.1N HCl except for A5 and A8.

The A5, coated for weight gain of 5% with mixture of ethyl cellulose dispersion and Opadry II at ratio of 50:50 w/w of total solids (i.e., hydrophobic cellulose to hydrophilic polymers) failed to meet the dissolution criteria compared to A2 which had same core composition.

Similarly, A8 Coated for weight gain of 1.5% with ethyl cellulose coating dispersion (hydrophobic cellulose) retarded the release and it doesn't meet the criteria of reference dissolution limits of 0.1N HCL established in table 1.

The percentage release of mesalamine at 4.5 pH at various time intervals is herewith provided in Table 6.

TABLE 6

| Time | | | | | | | | | | | | | |
|---|---|---|---|---|---|---|---|---|---|---|---|---|---|
| (h) | A1 | A2 | A3 | A4 | A5 | A6 | A7 | A8 | A9 | A10 | A11 | A12 | A13 |
| 1 | 6 | 8 | 7 | 5 | 2 | 7 | 10 | 5 | 9 | 8 | 7 | 32 | 14 |
| 2 | 12 | 13 | 12 | 10 | 3 | 11 | 16 | 7 | 18 | 12 | 12 | 59 | 24 |
| 4 | 20 | 24 | 21 | 20 | 5 | 27 | 27 | | 28 | 24 | 21 | 91 | 39 |
| 6 | 30 | 32 | 27 | 29 | | 37 | 34 | | 38 | 31 | 30 | 100 | 46 |
| 8 | 40 | 38 | 35 | 38 | | — | 40 | | 45 | 37 | 38 | | 60 |
| 12 | 59 | 50 | 46 | 56 | | 67 | 59 | | 55 | 55 | 56 | | 85 |

From table 6 (A1-A11) it can be inferred that following trials failed to release drug in pH 4.5 as per the dissolution limits in Table 1
a. A3 coated with weight gain of 4.2% with mixture of ethylcellulose dispersion and Opadry II at ratio of 1:3 w/w of total solids (i.e., hydrophobic cellulose to hydrophilic polymers) has completely failed to release drug due to curing process.
b. A5 coated for weight gain of 5% with mixture of ethyl cellulose dispersion and Opadry II at ratio of 1:1 w/w of total solids (i.e. hydrophobic cellulose to hydrophilic polymers)
c. A6 Coated with weight gain of 1% w/w with ethyl cellulose dispersion (hydrophobic cellulose) has failed to control the release in pH 4.5 at end time points.
d. A8 Coated with weight gain of 1.5% w/w with ethyl cellulose dispersion (hydrophobic cellulose) has retarded the release in pH 4.5.
e. A10 coated for weight gain of 12% w/w with mixture of ethyl cellulose dispersion and Opadry II at ratio of 2:4 w/w of total solids (i.e. hydrophobic cellulose to hydrophilic polymers) has failed to match the dissolution limits.

The percentage release of mesalamine at 7.5 pH at various time intervals is herewith provided in Table 7.

TABLE 7

| T | | | | | | | | | | | | | |
|---|---|---|---|---|---|---|---|---|---|---|---|---|---|
| (h) | A1 | A2 | A3 | A4 | A5 | A6 | A7 | A8 | A9 | A10 | A11 | A12 | A13 |
| 1 | 18 | 23 | 21 | 16 | 3 | 22 | 39 | 12 | 27 | 25 | 18 | 81 | 28 |
| 2 | 37 | 39 | 38 | 31 | 9 | 38 | 60 | 20 | 54 | 37 | 38 | 100 | 48 |
| 4 | 76 | 63 | 74 | 67 | 12 | 69 | 85 | | 80 | 66 | 76 | | 72 |
| 6 | 97 | 81 | 93 | 85 | | 86 | 88 | | 93 | 78 | 89 | | 85 |
| 8 | | | 99 | 95 | | 95 | | | 97 | 84 | 94 | | 100 |
| 12 | | | | 100 | | 100 | | | | 92 | 100 | | |

From table 7 it can be inferred that, A5 coated for weight gain of 5% with mixture of ethyl cellulose dispersion and Opadry II at ratio of 1:1 w/w of total solids (i.e., hydrophobic cellulose to hydrophilic polymers) and A8 Coated for weight gain of 1.5% with ethyl cellulose dispersion (hydrophobic cellulose) retarded the release and it doesn't meet the criteria of reference dissolution limits of pH 7.5 established in table 1 similar as that of 0.1N HCl.

Further, from a combined reading of Table 5-table 7 it can be concluded that, the coating hydrophobic cellulose above 1% completely retards the release and failed to meet the dissolution criteria set in Table 1. Similarly, the mixture of hydrophobic cellulose and hydrophilic polymers coating fails to meet the dissolution criteria at weight gain 12% w/w to the core weight. From the results, we can infer that not all compositions pass the dissolution criteria. It appears that the weight ratios of the weight ratio of hydrophobic cellulose and hydrophilic polymer mixture (ethyl cellulose to Opadry) in desired weight percentage plays a critical role in release of the active ingredient 5-ASA. Those compositions where the weight ratio of the was beyond the hydrophobic cellulose and hydrophilic polymer mixture (ethyl cellulose to Opadry) the claimed ranges i.e., 1:9 to 4:6, and where the weight gain was beyond the claimed range (2%-10% to the total weight of minitablet), such compositions failed to meet the dissolution criteria. The mixture composition also retards the drug release on curing coated minitablets.

### Advantages of the Present Disclosure:

The present disclosure provides a pharmaceutical composition and process for preparing high drug load of 5-ASA as controlled release minitablets using a pharmaceutically acceptable diluent or carrier for oral administration. The pharmaceutical composition comprises the minitablets as core, and a coating layer; the core tablet comprising: i) 5-ASA or its pharmaceutically acceptable salt, solvate, and/or ester thereof having a weight percentage in a range of 80%-90% w/w with respect to the core weight of the core mini tablet; and a coating layer comprising: i) hydrophobic cellulose to the weight gain of 0.5% to 1%; or ii) hydrophobic cellulose and hydrophilic polymer mixture, coated to weight gain of 2%-10% to the total weight of the core minitablet, such that the total weight of the controlled release composition is in the range of 595-650 mg. The composition of the present disclosure is a mini-tablet formulation with high drug load and functional coating, so that single dosage unit encapsulating the multiple minitablets can be administered in size smaller than the currently available product size of 00 EL, thereby enhancing the swallowability of the dosage unit. Further, the ease of swallowing the dosage unit ensures patient compliance, thereby overcoming the drawbacks of the prior art.

1. A controlled release composition comprising:
a. a core minitablet comprising: i) 5-ASA or its pharmaceutically acceptable salt, solvate, and/or ester thereof having a weight percentage in a range of 80%-90% w/w with respect to the weight of the core mini tablet; and
b. a coating layer comprising: i) hydrophobic cellulose to the weight gain of 0.5% to 1% w/w; or ii) hydrophobic cellulose and hydrophilic polymer mixture, coated to weight gain of 2%-10% w/w to the weight of the core minitablet, such that the total weight of the controlled release composition is in the range of 595-650 mg.

2. The controlled release composition as claimed in claim 1, wherein ratio of the hydrophobic cellulose to hydrophilic polymers is in the range of 1:9 to 4:6.

3. The controlled release composition as claimed in claim 1, wherein ratio of the hydrophobic cellulose to hydrophilic polymers is 1:3.

4. The controlled release composition as claimed in claim 1, wherein ratio of the hydrophobic cellulose to hydrophilic polymers is 4:6.

5. The controlled release composition as claimed in claim 1, wherein ratio of the hydrophobic cellulose to hydrophilic polymers is 1:4.

6. The controlled release composition as claimed in claim 1, wherein the core minitablet further comprises: binder(s) having a weight percentage in a range of 1%-10% with respect to the core minitablet; diluent(s) having a weight percentage in the range of 0.5%-20% with respect to the core minitablet, disintegrant(s) having a weight percentage in a range of 1%-10% with respect to the core minitablet, lubricant(s) having a weight percentage in a range of 1%-5% with respect to the core minitablet, glidant(s) having a weight percentage in a range of 1%-5% with respect to the core minitablet, and retarding agent(s) having a weight percentage in the range of 4-18% with respect to the core minitablet.

7. The coating layer composition in claim 1, wherein the hydrophobic cellulose is in the range of 0.5-1%

8. The controlled release composition as claimed in claim 6, wherein the diluents are selected from a group consisting of lactose, lactose monohydrate, starch, hydrolysed starch, partially hydrolysed starch, stearic acid, microcrystalline cellulose, calcium phosphates, mannitol and/or any co-processed excipients of above; wherein the binder are selected from a group consisting of polyvinyl pyrrolidine, hydroxypropyl cellulose, hypromellose, acacia, starch, starch derivatives and gelatin; the disintegrant(s) is selected from a group consisting of crospovidone, sodium starch glycolate, starch, hydrolysed starch, partially hydrolysed starch, croscarmellose sodium, the lubricant(s) are selected from a group consisting of magnesium stearate, calcium stearate, stearic acid, glyceryl dibehenate, glyceryl stearate, other glyceride derivatives, low MW polyethylene glycols, the glidant(s) are selected from the group consisting of colloidal silicon dioxide, silicon dioxide and talc, and the retarding agent(s) are selected from a group consisting of ethyl cellulose, magnesium stearate, calcium stearate, glyceryl monostearate, glyceryl dibehenate, glyceryl distearate, stearic acid and any stearic acid or fatty acid derivatives thereof.

9. The coating layer composition as claimed in claim 1, wherein the hydrophobic cellulose is ethyl cellulose.

10. The controlled release composition as claimed in claim 1, wherein the core is optionally coated with a coating solution comprising seal coating polymers to bring about 0.3%-5% weight gain to the core minitablet.

11. The controlled release composition as claimed in claim 1, wherein the controlled release layer is coated on the core minitablet or a seal coated surface.

12. A method of preparing the controlled release, the method comprising: (a) mixing/granulating 5-ASA or premixed 5-ASA and retarding agent along with a pharmaceutically acceptable binder solution prepared in suitable solvents (e.g. water or ethanol or Isopropyl alcohol or mixture of two or more Solvent) to obtain a first mixture; b) drying the first mixture to a temperature range of 40-70° C. to obtain a dried mixture; c) passing the granules through mill and/or sifter of mesh range between mesh size #16 and mesh #60, wherein the granules have a size in the range of 100 microns to 700 microns; d) mixing the granules obtained in step c) with other diluents, retarding agents, glidants and lubricants to obtain free flowing granule of 5-ASA; e) compressing the granules obtained in step d) using single tip or multi-tip (of about 16 tips) and die to obtain the core minitablet of claim 1; and f) coating the core minitablet with a coating solution comprising: i) hydrophobic cellulose to the weight gain of 0.5% to 1%; or ii) hydrophobic cellulose and hydrophilic polymer mixture, coated to weight gain of 2%-10% to the total weight of the core minitablet, such that the total weight of the controlled release composition is in the range of 595-650 mg.

13. A method to improve patient compliance comprising administering to a patient in need thereof the composition of claim 1.
